(19) United States
(12) Patent Application Publication
(10) Pub. No.: US 2023/0117926 A1
(43) Pub. Date: Apr. 20, 2023

(54) CLEANING DEVICE AND CLEANING SYSTEM FOR A BLOOD TREATMENT DEVICE

(71) Applicant: FRESENIUS MEDICAL CARE DEUTSCHLAND GMBH, Bad Homburg (DE)

(72) Inventors: Winfried BREHM, Hofheim (DE); Rafael STERZER, Schweinfurt (DE); Paul SEIT, Schweinfurt (DE)

(73) Assignee: FRESENIUS MEDICAL CARE DEUTSCHLAND GMBH, Bad Homburg (DE)

(21) Appl. No.: 17/908,245

(22) PCT Filed: Feb. 22, 2021
(86) PCT No.: PCT/EP2021/054281

§ 371 Date: Aug. 31, 2022

(30) Foreign Application Priority Data
Mar. 11, 2020 (DE) 10 2020 106 593.0

(51) Int. Cl. A61M 1/16; F16H 25/20

(52) U.S. Cl. CPC A61M 1/168; F16H 25/20; A61M 2209/10

(57) ABSTRACT

The invention relates to a cleaning device, which has a base body (1), a first and a second line element (2a, 2b), which extend perpendicularly in the base body (1) from a first surface of the base body (1). In addition, the cleaning device has a first through hole (3a), wherein the first through hole (3a) extends at a first point of the first line element (2a) spaced apart from the first surface of the base body (1) to a first lateral surface of the base body (1), which differs from the surface of the base body (1), a first connecting element (31a), which extends from the base body (1) along the first through hole (3a) from the first lateral surface of the base body (1), so that the middle line of the through hole (3a) further extends from the first lateral surface of the base body (1) through the first connecting element (31a).

### TECHNICAL FIELD

The present invention relates to a cleaning device, a cleaning system, as well as a use thereof in the case of a blood treatment device, in particular a dialysis machine.

### BACKGROUND

The term blood treatment device is to be understood, inter alia, as a dialysis machine. Dialysis machines are frequently used in dialysis centers to treat chronic kidney disease.

During the dialysis, blood of the patient flows continuously through the blood chamber of the dialyzer, while dialysis fluid flows continuously through the dialysis fluid chamber. Pre-made dialysis fluid concentrates, which are diluted with water in the dialysis devices, can be used to produce dialysis fluid. In dialysis centers, dialysis fluid concentrates are provided either as pre-made product in canisters, bags or cartridges or are provided from a central tank via a ring circuit system.

Centrally provided dialysis fluid concentrates can be handled easily by the user, they have the disadvantage, however, that the dialysis fluid cannot be individually adapted to the patient’s needs. Even though concentrates, which are provided in a decentralized manner, allow for an individual adaptation of the dialysis fluid to the patient, they have to be brought to the dialysis device for each individual dialysis treatment.

Bags or cartridges, which are filled with a powdery dialysis fluid concentrate, are used in the dialysis for the production of a liquid dialysis concentrate. The concentrate bags or cartridges contain an amount of powdery dialysis fluid concentrate, which is sufficient for a single dialysis treatment. The bags or cartridges are filled with bicarbonate. Commercially available bicarbonate bags contain 650 to 950 g of sodium bicarbonate. First of all, a liquid bicarbonate concentrate is produced from the powdery bicarbonate concentrate. To produce the dialysis fluid, a further acid concentrate is required, which is provided in a canister or by means of a central supply. Bicarbonate concentrate and acid concentrate are then mixed with water to form the final dialysis fluid.

The dialysis machine thus further has a water supply, via which pure water is supplied. The desired mixing of pure water with the corresponding concentrate then takes place in the dialysis machine.

The provision of the liquid concentrate via concentrate containers is associated with different challenges, because the concentrate containers are heavy and because the handling thereof is thus complex for the nursing staff. In addition, suction wands of the dialysis machine have to be introduced into the concentrate containers in order to provide the concentrate and have to be inserted into the dialysis machine again after a treatment for cleaning purposes. This handling of the concentrate containers is thus time-consuming and may lead to an extension of the breaks between the individual treatments.

In the alternative, the concentrate can also be supplied via a central supply and a corresponding central supply connection on the dialysis machine. This, however, requires corresponding clinic equipment and may be associated with regard to the flexibility of the available concentrate and the maintenance effort for the lines operated for this purpose.

A connector for connecting a concentrate bag, which is filled with dry concentrate and can be hooked to a dialysis machine, is known from EP 1 344 550 A1. In the case of this type of concentrate supply, certain steps are also necessary between the treatments. For instance, a contamination protection attached to the concentrate bag has to be removed manually, the concentrate bag has to be inserted into, that is connected to, the corresponding receptacle on the dialysis machine, and has to lastly be connected to the dialysate circuit.

In the case of the prior art, the connection of the concentrate bag mostly takes place from below. This means that the concentrate bag is attached to connecting spouts. After the treatment, the concentrate bag is lifted off the connecting spouts. Residual fluid, which is still present in the connection points of the concentrate bag, can thereby easily drip onto the floor.

However, residual fluid of the bicarbonate solution can also lead to deposits on the connecting spouts of the dialysis machine. This is caused by calcium and magnesium salts. Due to the formation of the connection points, which are open to the bottom, this residual fluid can drip into the connecting spouts and can cause deposits there. These deposits can lead to damages to the sealing rings assigned to the connection points.

After a treatment, the concentrate bag has to be disconnected and removed from the dialysate circuit. A cleaning program can only be started on the dialysis machine after the removal. The surface cleaning of the dialysis machine can also be performed only after the removal of the concentrate bag by the nursing staff.

In addition to high safety requirements, the aspect of time plays a special role for the dialysis treatment, in particular in dialysis centers.

Due to the fact that sufficient time is to be available for the actual treatment, in particular the time between the treatments is subjected to a constant time optimization. As described above, in addition to the preparation of the patient, the preparation of the dialysis machine for the next treatment also takes time between treatments due to steps, such as the provision of the required concentrate containers or the disinfection of the dialysis machine.

The present application is thus based on the object of providing a cleaning device for a blood treatment device, which allows for an improved handling and/or time savings. In addition, the patient’s safety is to be ensured by means of a simpler cleaning of the blood treatment device.

### SUMMARY OF THE INVENTION

The object on which the invention is based is solved by means of the cleaning device for a blood treatment device according to claim 1 as well as by the cleaning system for a blood treatment device according to claim 10.

Advantageous further developments and embodiments are subject matter of the dependent claims.

According to the invention, a cleaning device for a blood treatment device is provided, which is provided a base body, a first and a second line element, which extend in the base body from a first surface of the base body, preferably perpendicularly. The cleaning device can further have a first through hole. The first through hole can extend from a first point of the first line element spaced apart from the first surface of the base body to a surface of the base body, which differs from the first surface of the base body. The first through hole can thus extend a fluidic connection from the first line element to an outer side of the cleaning device. The through hole can likewise extend to an inner side of the second line element.

The cleaning device can further have a first connecting element, which extends from the base body, preferably along the center line, of the first through hole from the first lateral surface of the base body, so that the through hole further extends from the first lateral surface of the base body through the first connecting element. The connecting element can thereby be formed integrally with the cleaning device or can be pressed, adhered or screwed into it. The connecting element can thereby be made of a different material. The base body can be made, for example, of metal, and the connecting element of a flexible material, for example plastic. The first connecting element can thereby be a protrusion, which is preferably formed cylindrically, conically or step-shaped and extends from the base body, preferably essentially perpendicularly.

According to the invention, a cleaning device for a blood treatment device is a device, which can be used to clean the blood treatment device, in particular a dialysis machine. In particular the connecting spouts, which are provided for the connection of a concentrate container to the dialysis machine, can be cleaned by means of the cleaning device. Due to the fact that the connecting spouts can be introduced into the cleaning device from the top, it is possible to prevent a dripping of cleaning fluid onto the floor and in particular into the connecting spouts.

At a first point, the first through hole arranged in the base body of the cleaning device extends out of the first line element, which is spaced apart from the surface. The line elements are formed, for example, as blind holes or blind bores. The through hole can in particular extend from the inner side of the first line element in such a way that fluid in the first line element can easily flow through the through hole and can thus easily flow out of the line element. In other words, the through hole can be formed in such a way that it extends away from the underside of the line element.

According to a further development of the cleaning device, a connecting duct between the first and the second line element can be formed at a second point of the line elements, which is spaced apart from the surface of the base body. The cleaning device can additionally have recesses, which differ from the line elements. These recesses can allow for a fluidic connection between a concentrate bag attached to the cleaning device and connecting spouts of a dialysis machine.

The connecting duct can thus establish a fluidic connection between the line elements. The connecting duct can be formed close to a bottom area of the line elements or extends away from the bottom area on one plane with its underside. The connecting duct can extend essentially perpendicular to the alignment of the through hole.

According to a further development of the cleaning device, the first line element can extend to a first lateral surface of the base body. At a third point, which is spaced apart from the first surface of the base body, the second line element can further have a second through hole to the first lateral surface of the base body, which differs from the first surface of the base body.

The second through hole can thereby be formed parallel to the first through hole in the base body. In accordance with the first through hole, the second through hole can thereby be a through bore, which connects an outer side, which can also correspond to the underside of the base body to the second line element. Fluid can in particular flow out of the second line element through the second through hole. The second through hole is thus formed on one plane with the underside of the second line element or is formed close to the underside. However, the first and second through hole can also slope away from the connection point with the first and second line element away from the surface of the base body, so that fluid can easily flow out of the first and second line element.

According to a further development of the cleaning device, at least one blocking element can be attached in the first and second line element. A fluidic connection between the connecting duct to the first and second through hole can be prevented therewith. The blocking element can additionally have predetermined breaking points, for example due to a reduced wall thickness in a partial area.

The blocking element can be a circular plastic element, which is in each case formed on the inner circumference of the line elements. In addition, the blocking element itself can have predetermined breaking points, which break through the blocking element when connecting spouts move into the line elements, so that a fluidic connection between the connecting spouts and the through holes can be formed. In the alternative or in addition, the blocking element can in each case also have predetermined breaking points in the fastening on the inner circumference. The predetermined breaking points can be perforated curved portions of the blocking element, so that the wall thickness of the blocking elements is formed to be thinner at these points. In the alternative, the blocking element can be formed between the line elements in the connecting duct.

According to a further development of the cleaning device, the base body can be formed of a first base element and a second base element. The first and second base element can thereby in each case have a groove, which has a semicircular cross section, for forming the connecting duct, and the first and second base element can in each case have two recesses, which have a semicircular cross section, for forming the first and second line element each when connecting the first and second base element.

To form the connecting duct and the line element, the first and second base element can thereby be firmly connected to one another or so that they can be separated again, in other words screw-connected, adhered or welded. To form the line elements as well as the connecting duct in a fluidically tight manner, a sealing contour can be formed between the first and second base element.

According to a further development of the cleaning device, the base body can be formed of a first base element and a second base element, and the first base element can have two blind bores and the second base element can have two through bores, the diameter of which corresponds to the blind bores, for forming the first and second line element, the first base element and second base element can additionally have a groove, which has a semicircular cross section, for forming the connecting duct.

According to a further development of the cleaning device, the latter can further have a first cylindrical receptacle comprising at least one, preferably two, through openings, and a second cylindrical receptacle comprising a through opening. In addition, a cylindrical appendage can in each case be provided on the through openings, and have at least one, preferably two, tubular connecting elements, which can be attached to the appendages and which can be introduced into the connecting duct and/or the through hole in such a way that they connect the receptacles received in the line elements. In addition, the first through hole and/or the connecting duct can be open towards the first surface, so that the cylindrical receptacles as well as the tubular connecting elements can each be placed into the base body.

According to a further development of the cleaning device, the first and second line element can run parallel to one another in their center line and/or the first and the second through hole can run parallel to one another in its center line.

According to a further development of the cleaning device, a middle element can be formed between the first and second line element. The middle element can thereby be formed in a curved manner and extends away from the first surface, preferably perpendicularly. A middle portion between the first and second line element can thus be formed to be open.

According to a further development of the cleaning device, at least one sealing shell can be formed in the first and/or second line element, wherein the sealing shell is cylindrical and can have at least one lateral passage opening, the diameter of which corresponds to the connecting duct and/or to the through hole and can be formed on the jacket surface of the sealing shell.

According to the invention, a cleaning system for a blood treatment device, in particular a dialysis machine, is formed, and can have a housing for connection to a blood treatment device, in particular a dialysis machine, a cleaning device, preferably according to one of the preceding aspects, which is movably arranged in the housing. In addition, a first closing element can be formed, to which a fluid flow from the first line element can be connected via the first connecting element, and/or a second closing element, to which a fluid flow from the second line element can be connected via the second connecting element. In addition, a drive element for moving the cleaning device can be formed. The drive element can thereby be arranged in the housing or outside of the housing. The drive element can be an electric motor or can be a hydraulic or pneumatic drive. In addition, a sensor for capturing a movement of the cleaning device can be arranged, preferably in the housing.

According to a further development of the cleaning system, the sensor can be a light barrier or a Hall sensor. The light barrier can be a printed circuit board, preferably attached above the cleaning device.

According to a further development of the cleaning system, at least one guide rail formed in the housing, preferably integrally, on which the cleaning device can be shifted, can have. The first drive element can further have a spindle nut and an electric motor comprising a threaded spindle.

According to a further development of the cleaning system, the threaded nut can be formed on the middle element of the cleaning device, which is preferably formed in a curved manner and can have a different material than the base body, or the middle element, which is preferably formed in a curved manner, can have a protrusion, which is formed in the direction of the light barrier, so that a triggering of the light barrier can be attained in response to a moving, in particular a shifting of the cleaning device.

According to a further development, a use of a cleaning system according to a preceding aspect in the case of a blood treatment device, in particular a dialysis machine, is provided.

According to a further development, a use of a cleaning device according to one of the preceding aspects in the case of a blood treatment device, in particular a dialysis machine, is provided.

By means of the cleaning device as well as cleaning system, it is possible to attach a concentrate bag to the blood treatment device, while a cleaning step of the connecting spouts still takes place. Due to the formation of the cleaning device, it is additionally possible to ensure the safety of the patient. Fluid can in particular be prevented from dripping out of a concentrate bag, which is connected to the blood treatment device, in particular dialysis machine.

The above-described features and functions of the present invention as well as further aspects and features will be described in more detail below on the basis of a detailed description of preferred embodiments with reference to the enclosed figures. Identical features/elements and features/elements comprising the same function are identified with the same reference numerals in the figures.

### BRIEF DESCRIPTION OF THE DRAWINGS

In which

FIG. 1 shows a schematic diagram of a cleaning device according to a first embodiment;

FIGS. 2a, 2b show a schematic use of the cleaning device according to the first embodiment;

FIG. 3 shows a schematic diagram of the cleaning device comprising a blocking element;

FIG. 4 shows a schematic diagram of the cleaning device according to a second embodiment;

FIG. 5 shows a schematic diagram of the cleaning device according to a third embodiment;

FIG. 6 shows a schematic diagram of the cleaning device according to a fourth embodiment;

FIG. 7 shows a schematic diagram of the cleaning device according to a fifth embodiment;

FIG. 8 shows a schematic diagram of a cleaning shell for a cleaning device;

FIG. 9a shows a schematic illustration of a cleaning device according to a sixth embodiment;

FIG. 9b shows a schematic diagram of the cleaning device according to the sixth embodiment for use in a blood treatment device,

FIG. 10 shows a schematic diagram of a cleaning device according to a seventh embodiment for use in a blood treatment device;

FIGS. 11a, b show a schematic diagram of the cleaning device according to the seventh embodiment for use in a blood treatment device;

FIGS. 12a, b show a schematic diagram of the cleaning device according to an eight embodiment;

FIGS. 13a, b show a schematic diagram of a use of the cleaning device according to the eighth embodiment;

FIG. 14 shows a schematic diagram of a cleaning device according to a ninth embodiment;

FIG. 15 shows a schematic illustration of a cleaning system;

FIG. 16 shows a housing cover and a housing portion, and

FIG. 17 shows a schematic illustration of connecting spouts for introduction into a cleaning device.

### DETAILED DESCRIPTION OF AN EXEMPLARY EMBODIMENT

With reference to FIG. 1, a first embodiment will be described below. FIG. 1 thereby shows a cleaning device comprising a base body 1. The base body 1 further has a first and second line element 2a, 2b, which are formed as hollow cylinder here and each have a bottom plate on an underside of the first and second line element 2a, 2b and are thus fluidically closed on an underside. Each of the two line elements 2a, 2b has a through hole 3a, 3b, which is formed on the line elements 2a, 2b on a side, which differs from the surface side. The line elements 2a, 2b are formed to run parallel to one another here.

The through holes 3a, 3b are further formed at an equal distance from the surface. The connecting elements 31a, 31b in each case extend parallel to one another from the through holes 3a, 3b. In the embodiment illustrated in FIG. 1 here, the connecting elements 31a, 31b lead to an adapter hollow cylinder located between the line elements 2a, 2b. A bag comprising bicarbonate concentrate can be fastened to this adapter hollow cylinder. The concentrate bag can have a flexible material, in particular of PVC, PVT or polyethylene and can be fused to the base body 1. A connecting duct 5 runs between the line elements 2a, 2b above the connecting elements 31a, 31b, that is, closer to the surface of the base body 1. The connecting duct 5 fluidically connects the line elements 2a, 2b, so that a fluid, which flows in from the surface side, can flow via the first line element 2a and the connecting duct 5 to the second line element 2a, 2b and from there out of the base body 1.

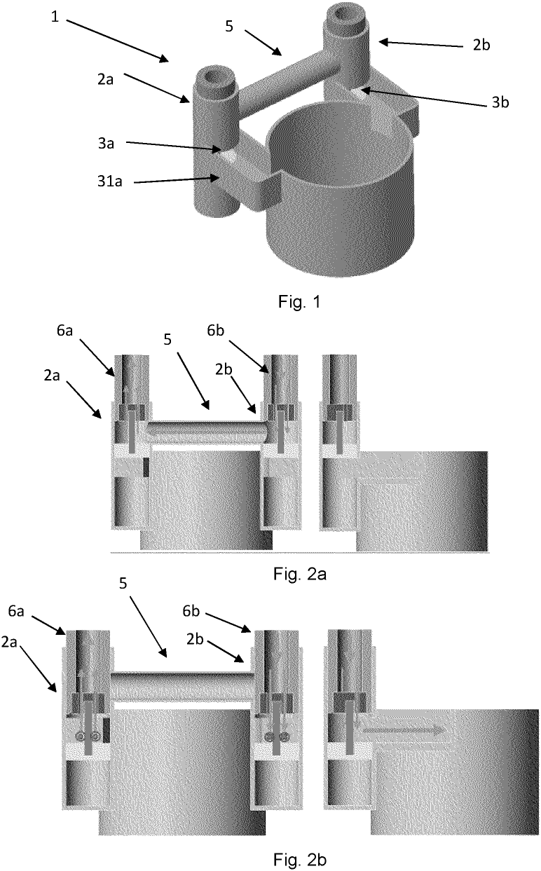

If this cleaning device is used during a blood treatment, in particular a dialysis treatment, a cleaning step can take place in this cleaning device. As shown in FIG. 2a, connecting spouts 6a, 6b, as shown in FIG. 17, which are fluidically connected to the dialysis machine, are moved into the line elements 2a, 2b from the surface of the base body 1 for this purpose. For this purpose, linear guides can be provided on the inner surface areas of the line elements 2a, 2b, in which the connecting spouts 6a, 6b can be moved in a linearly shiftable manner. In the alternative, the inner surfaces can be formed to be smooth, so that the connecting spouts 6a, 6b can be linearly shifted. In the alternative, internal threads can be arranged in the line elements, so that the connecting spouts 6a, 6b are introduced by means of a rotational movement.

The sequence of operations for cleaning the connecting spouts by means of the cleaning device begins with an attaching of the cleaning device, to which a concentrate bag is attached, to a dialysis machine. The connecting spouts 6a, 6b of the dialysis machine move into the line elements 2a, 2b. A stamping device, which is shown in FIG. 2, is thereby attached to the connecting spouts 6a, 6b. If the connecting spouts move into the line elements 2a, 2b in a first, upper position, as shown in FIG. 2, the punching elements close the line elements 2a, 2b below. In other words, the top side of the punching elements is located in one plane with the underside of the connecting duct 5. Flowing out of the connecting spouts 6a, 6b, fluid can thus not flow to the through holes 3a, 3b or connecting elements 31a, 31b.

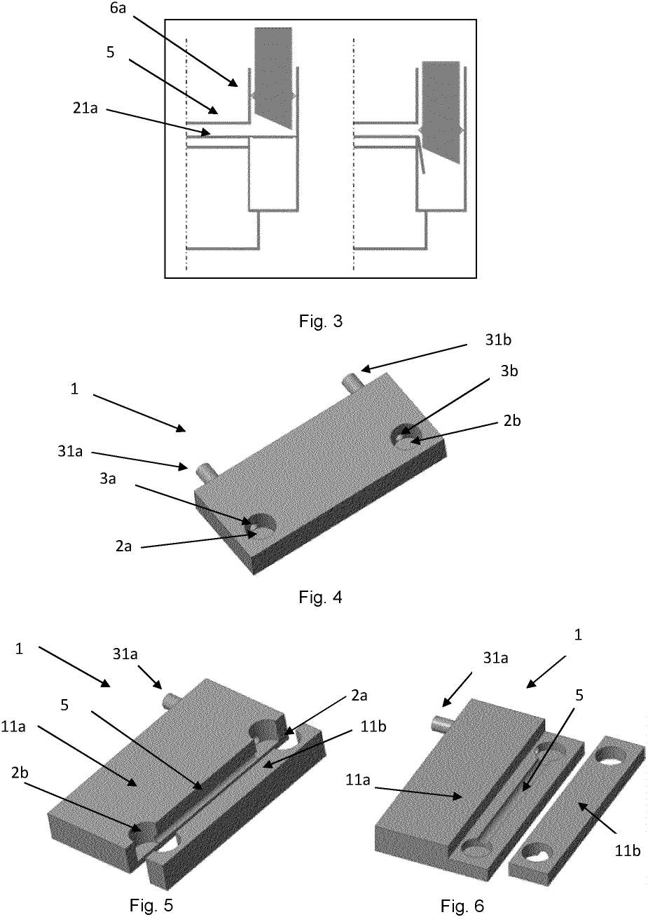

In this cleaning position, however, a fluid flow between the connecting spouts can take place via the connecting duct 5. If the connecting spouts 6a, 6b move further into a position shown in FIG. 2b, away from the surface side of the base body 1 within the line elements 2a, 2b, the punching elements ultimately reach to a position below the through holes 3a, 3b. In this position, fluid can flow out of the connecting spouts 6a, 6b via the connecting elements 31a, 31b into the concentrate bag, whereas a fluid flow is fluidically closed by the connecting duct 5 via lateral surfaces of the connecting spouts 6a, 6b. In the alternative, FIG. 3 shows a schematic diagram of the cleaning device comprising a blocking element 21a. The blocking element 21a is arranged in the line element 2a, 2b in such a way that it allows a fluidic connection between the line elements 2a, 2b via the connecting duct 5, but prevents a fluidic connection of the connecting duct 5 to the connecting elements 31a.

The blocking element 21a is advantageously in each case formed as circular blocking element 21a in line elements 2a, 2b below the lower edge of the connecting duct 5. If the connecting spouts 6a, 6b are moved into the second position, that is, into a position in the line elements 2a, 2b, in which a fluidic connection to the concentrate bag is formed, and the lateral surfaces of the connecting spouts 6a, 6b thus close the connecting duct 5, the connecting spouts 6a, 6b break through the blocking elements 21a in order to get into this position. For this purpose, the blocking elements 21a can have predetermined breaking points and can, for this purpose, have a smaller wall thickness, for example in a partial portion or can be formed to be weakened in a partial portion.

FIG. 4 shows a schematic diagram of the cleaning device according to a second embodiment. The line elements 2a, 2b extend perpendicular to a surface of the base body 1 here and are formed as blind holes or blind bores. On a side, which is spaced apart from the surface, in particular directly adjacent to a bottom area of the line elements 2a, 2b, the through holes 3a, 3b are formed here. They extend parallel within the base body 1 to a lateral surface here. The connecting elements 31a, 31b are formed adjacent to these through holes 3a, 3b. They protrude essentially perpendicularly from the surface of the base body 1. If connecting spouts 6a, 6b, as illustrated in FIG. 17, are moved into the line elements 2a, 2b when using the cleaning device according to the second embodiment, said connecting spouts can be flushed in the line elements 2a, 2b. Fluid thereby flows out of a connecting spout 6a, 6b into one of the line element 2a, 2b and from a line element 2a, 2b via the connecting duct 5 to the other line element 2a or 2b, respectively. During this flushing process, the connecting elements 31a, 31b are closed, for example, by means of a closing elements, which can be brought into contact therewith.

The fluid thereby flushes around the connecting spouts 6a, 6b. After this cleaning process, the fluid, which remains in the line elements 2a, 2b, can be suctioned off via the through holes 3a, 3b and adjacent connecting lines. It is advantageous for this purpose when a lower edge of the through holes 3a, 3b is adjacent to the underside of the line elements 2a, 2b. FIG. 5 shows a schematic diagram of the cleaning device according to a third embodiment. The base body 1 is thereby formed of a first base element 11a and a second base element 11b, so that a plate results in response to a connecting, for example screw-connecting, adhering or fusing. The connecting duct 5 is thereby created only after a connecting of the base elements, whereby a negative in the form of a recess with a semicircular cross section is formed here on each base element.

Along their longitudinal axis, these recesses extend essentially perpendicularly to the recesses of the line element 2a, 2b, which are formed of recesses, which have a semicircular cross section. A through hole 3a is thereby formed only on the line element 2a. In the case of a use of the cleaning device with a dialysis machine, fluid can thereby flow in via a line element 2a, can flow through the connecting duct 5 to the second line element 2b, and from there can flow back again via the connecting duct 5. At the end of the cleaning, the remaining fluid can be removed via the connecting element 31a, which had previously been closed, for instance by a valve, which is now open.

FIG. 6 shows a schematic diagram of the cleaning device according to a fourth embodiment. Here, the base body 1 also has a first and a second base element 11a, 11b, which are connected to one another to form the connecting duct 5. The fluidically tight connection can also be attained here, for example by adhering, fusing or screw-connecting. The second base element 11b thereby has two through holes or through bores here, while the first base element 11a has blind holes, the diameter of which corresponds to the through holes. To form the connecting duct 5, the first as well as the second base element 11a, 11b has recesses, which each have a semicircular cross section, extending between the through bores or blind holes, respectively.

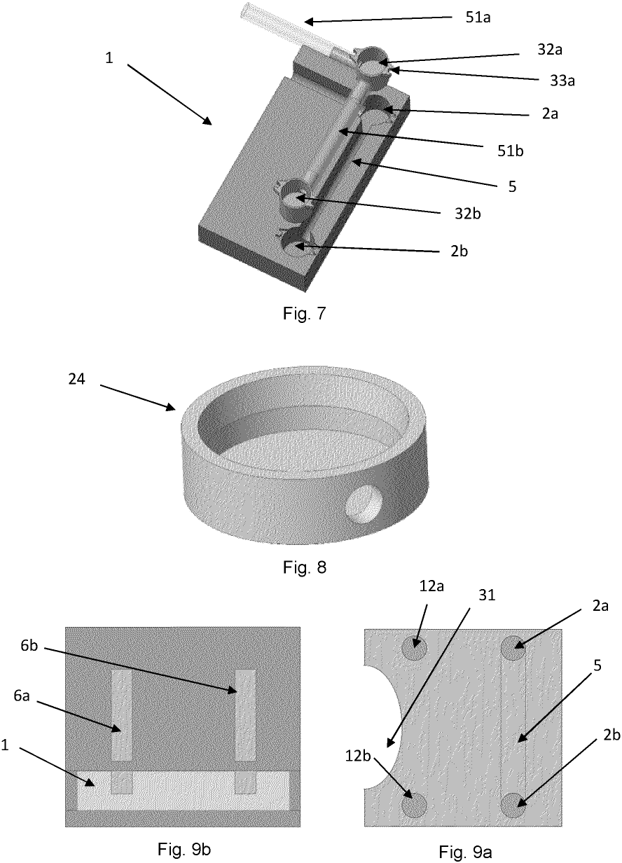

In this embodiment, a through hole 3a comprising connecting element 31a adjacent thereto is additionally also formed only on one line element 2a. The cleaning with connecting spouts 6a, 6b of a dialysis machine, which can be moved into the line elements 2a, 2b, takes place according to embodiment three. FIG. 7 shows a schematic diagram of the cleaning device according to a fourth embodiment. The cleaning device thereby further has a first cylindrical receptacle 32a, formed here as flushing pot comprising two through bores. In addition, the cleaning device has a second cylindrical receptacle 32b comprising a through bore. In their outer diameter, the flushing pots correspond to the inner diameter of the line elements 2a, 2b, so that they can be inserted into the latter.

In the inserted position, the through openings of the receptacles 32a, 32b are aligned with the connecting duct 5 formed in the base body 1 and with the through hole 3a. Connecting elements 51a, 51b, formed here as hoses, are mounted or attached, respectively, to appendages, which are provided on the receptacles 32a, 32b. In the case of this embodiment, the recesses in the base body 1 for forming the connecting duct 5 or the through hole 3a, respectively, do not have to be closed towards the surface of the base body 1. On the contrary, the connecting duct 5 and the through hole 3a through the connecting element 51a, 51b are formed in such a way that they are closed towards the surface. As illustrated in FIG. 7, threaded bores can be attached in the base plate, so that the receptacles, which have lateral tabs 33a, can be screw-connected in the base body 1. In the alternative, the receptacles can be fastened to the line elements 2a, 2b in the base body 1 by means of a press fit or can be adhered in the latter.

FIG. 8 shows a schematic diagram of a cleaning shell for a cleaning device, in particular for introduction into the line elements 2a, 2b. To be able to establish a fluidic connection, through bores are in each case formed in the cleaning shells, a through bore in the sealing shell 24 shown here. FIG. 9a shows a schematic illustration of a cleaning device according to a sixth embodiment. A concentrate bag can thereby be attached to the cleaning device in the receptacle 31. For this purpose, the concentrate bag can be in fluidic connection, for example with recesses 12a, 12b or can be pressed into them. The recesses 12a, 12b can be formed in the cleaning device and can allow a fluidic connection between a concentrate bag and the connecting spouts 6a, 6b. The cleaning device further has two line elements 2a, 2b, which are connected via a connecting duct 5. The cleaning device can thereby be arranged in a dialysis machine so as to be capable of being shifted in such a way that connecting spouts 6a, 6b of the dialysis machine, which are arranged so as to be vertically shiftable, can move into the line elements 2a, 2b for cleaning the connecting spouts 6a, 6b, and can move into the recesses 12a, 12b after a horizontal shifting of the cleaning device, so as to be fluidically connected to the concentrate bag.

If a concentrate bag hangs in the receptacle 31 provided for this purpose, the cleaning device moves horizontally into a device interior of the dialysis machine and positions the concentrate bag adapter below the connecting spouts in this way. These connecting spouts move vertically downward into openings of the concentrate bag and connect the bag for the dialysis operation in this way. As soon as the therapy has ended and the concentrate bag is emptied, the connecting spouts move vertically upward out of the concentrate bag adapter. The cleaning device then partially moves horizontally out of the dialysis device into the cleaning position. In the cleaning position, the connecting spouts move into the line elements 2a, 2b again. In this position of the cleaning unit, the concentrate bag hangs in front of the dialysis machine and can be removed. A new bag is hooked in again after the subsequent surface cleaning.

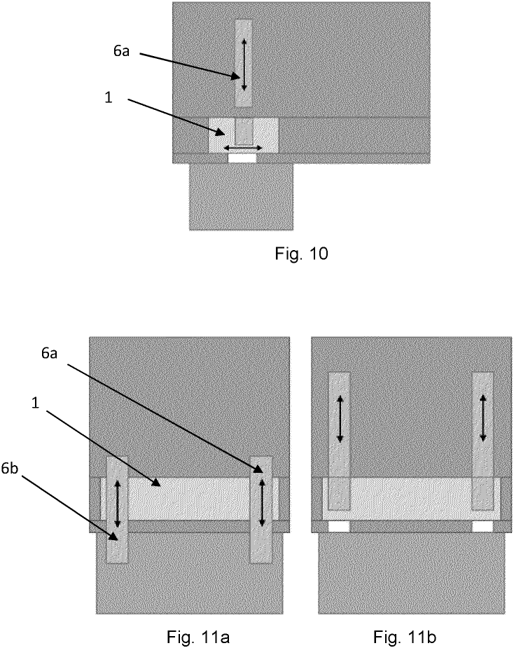

FIG. 10 shows a further schematic diagram for a use of a cleaning device on a dialysis machine. The nursing staff inserts the concentrate bag into a receptacle on a connector housing of the dialysis machine, in which the connecting spouts 6a, 6b are arranged. The receptacle is designed in such a way that the connecting openings in the concentrate bag are located exactly below the connecting spouts 6a, 6b. After ending the cleaning, the connecting spouts 6a, 6b move vertically upward out of the cleaning device. In response to their vertical downward movement, the connecting spouts 6a, 6b can connect the concentrate bag to a dialysate circuit through openings in the connector housing. After the therapy and the emptying of the concentrate bag, the connecting spouts 6a, 6b return into the upper end position. The cleaning device subsequently moves horizontally to below the connecting spouts 6a, 6b, so that they can move into the line elements 2a, 2b for the cleaning. The concentrate bag can now be removed and the surface disinfection can be performed. A new concentrate bag is subsequently inserted into the receptacle.

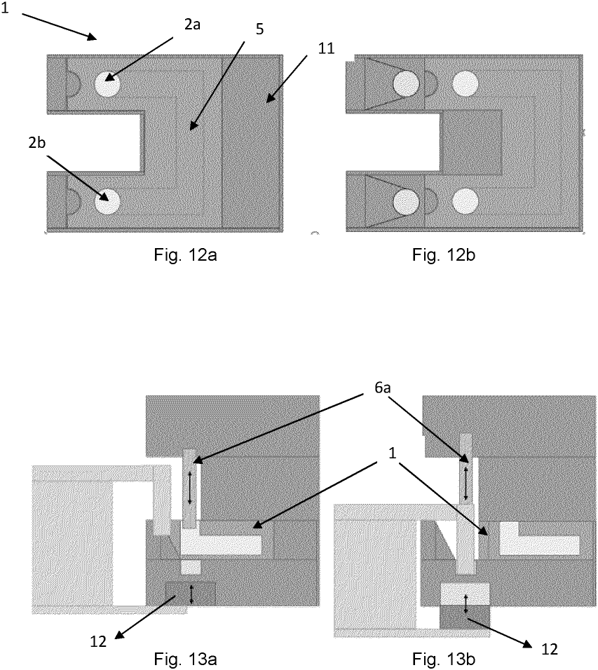

FIGS. 11a) and b) show the above-described arrangement from a front view with a connecting position FIG. 11a and a cleaning position FIG. 11b. FIG. 12 shows a schematic diagram of the cleaning device according to an eighth embodiment. In the case of this embodiment, the cleaning device has a 11 receiving unit for receiving a concentrate bag, wherein the cleaning device is formed to be movable, in particular shiftable, to the receiving unit. The connecting duct 5 runs between the line elements 2a, 2b, in a middle portion, wherein the connecting duct is formed to be U-shaped, so that a free space is formed between the line elements 2a, 2b. A certain relative position between cleaning device and receiving unit can be ensured by means of a control element, for example a spring, whereby the spring can be attached to the dialysis machine itself, so that the cleaning device always experiences a force, which brings it into the cleaning position.

The receiving unit has receptacles, for example receiving bores, for a concentrate bag, so that the concentrate bag is positioned in such a way that the connecting spouts 6a, 6b of the dialysis machine can be securely moved in said receptacles. In the case of this embodiment, the receiving bores are arranged for this purpose in the cleaning position so as to be aligned with the line elements 2a, 2b, while, when a concentrate bag is located in the receiving unit, the cleaning device is shifted in the connecting position in such a way that said concentrate bag is arranged below the connecting spouts. A sequence of a cleaning method will be described below. The start position of the cleaning device is thereby the cleaning position as shown in FIGS. 12 a, b. In this position, a concentrate bag can be hooked into the receiving unit. The positioning can be facilitated by means of bevels provided on the receiving unit, as well as by recesses formed in the cleaning device, for example semicircular grooves. In other words, the nursing staff can already hook a new concentrate bag to the receiving unit while the cleaning device is located in a front position, as shown in FIG. 12.

A press-fit element 12 shown in FIGS. 13 a and b can further formed on the underside of the receiving unit or of the housing 9, respectively. The press-fit element can be arranged in the receiving unit via a pre-loaded spring, that the concentrate bag is pushed into a position below the connecting spouts 6a, 6b. For this purpose, the press-fit element is pushed into the receiving unit only by an attaching of a concentrate bag. As soon as the connecting spouts 6a, 6b are moved out of the line elements 2a, 2b and the line elements 2a, 2b can thus be shifted, the press-fit element is pushed out of the receiving unit by means of the spring force and shifts the concentrate bag underneath the connecting spouts 6a, 6b. In other words, the two connecting spouts 6a, 6b move vertically upward out of the line elements 2a, 2b after the cleaning of the dialysate circuit. Following this, the press-fit element 12 moves out on the underside of the receiving unit and pushes the concentrate bag into the receiving bores along the bevel in this way.

In addition to the vertical direction, the movement along the bevel also includes a horizontal direction, whereby the cleaning device is horizontally shifted, so that the receiving bores become free. To connect the concentrate bag to the dialysate circuit, the connecting spouts 6a, 6b move vertically downward and connect the concentrate bag at the corresponding openings. After the therapy, the concentrate bag is emptied, the connecting spouts 6a, 6b move out of the concentrate bag, and the press-fit piece 12 moves back into the receiving unit. The nursing staff can then remove the concentrate bag. Due to the removal of the concentrate bag, the cleaning device returns into the cleaning position, so that the connecting spouts 6a, 6b can move vertically downward into the cleaning position again. The nursing staff performs the surface disinfection and hooks in a new concentrate bag during the dialysate circuit cleaning.

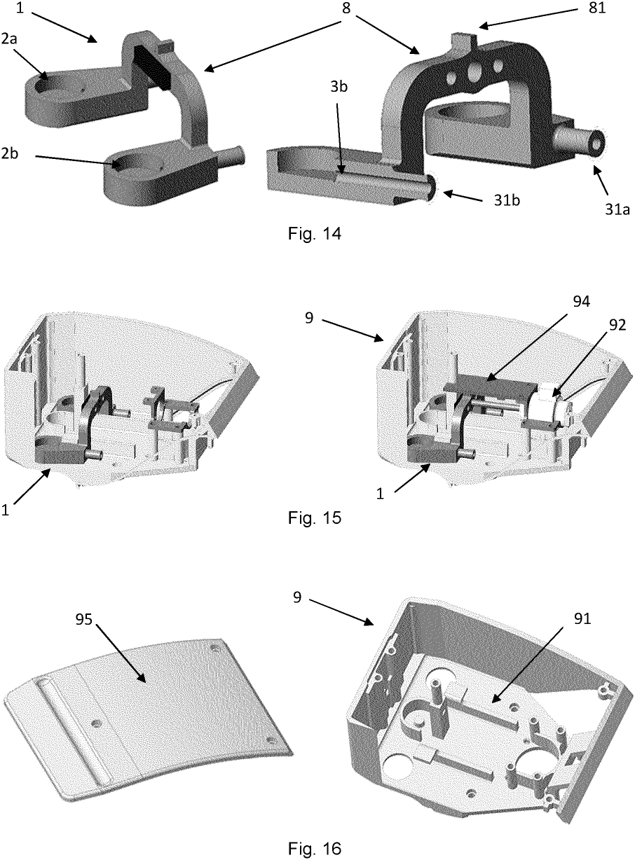

FIG. 14 shows a schematic diagram of a use of the cleaning device according to a ninth embodiment. The base body 1 thereby has two arm-like elements, in which the line elements 2a, 2b are formed. A middle element 8, which is curved in the opposite direction to the direction, in which the line elements 2a, 2b extend in the base body 1, is formed between these arm-like elements. Each of the two line elements 2a, 2b is connected to a through hole 3a, to which a respective connecting element 31a, 31b is adjacent. A bore for receiving a threaded nut can further be formed in the middle element 8. In the alternative, the threaded nut can be formed so as to be cut directly into the middle element 8.

In addition, a positioning element 81, which interacts with a sensor assigned to the dialysis machine and which can trigger a light barrier, for example as protrusion 81 from the middle element 8, can be formed on the middle element 8. FIG. 15 shows a schematic diagram of a cleaning system. A cleaning device according to the above-mentioned embodiment is arranged in a housing 9 here. To guide the cleaning device, guide rails 91 can be formed in the housing 9. The guide rails 91 can be two symmetrically arranged beams on the bottom of the housing 9. To prevent a tilting of the cleaning device, horizontal surfaces, which always cover an area of the cleaning device, can be located at one end of the beams.

To regulate a fluid flow, non-illustrated blocking elements, for example valves, can be formed on the connecting elements 31a, 31b. To move the cleaning device, a drive element is arranged in the housing 9. To capture the movement of the cleaning device, a printed circuit board is arranged in the housing 9 in such a way that the protrusion 81 on the middle element 8 can interact with forked light barriers, which are provided on the printed circuit board 94. In this embodiment, the drive element is an electric motor 92, which drives a threaded spindle, which engages with the threaded nut on the middle element 8. The housing 9 can further have a cover 95, which can be screw-connected to the housing 9 after the assembly of the elements.

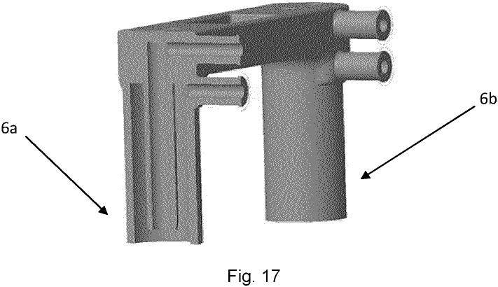

The housing 9 can further have a cover 95, a sealing element between the dialysis device and the housing 9, as well as a fastening adapter. To more easily position the cover 95 on the housing 9, positioning aids in the form of stops, as shown in FIG. 16, are located on an underside, on a front side, as well as on two further sides.

After the insertion of the cover 95, the latter is screw-connected to the housing 9 at three points. A rubber stopper in the color of the cover 95 is mounted to the screwed-in screw heads. They are to close the bores, so that the cover 95 has a plane surface area. As shown in FIG. 16, the groove on the upper side of the cover 95 serves to receive the concentrate bag on the housing 9. Due to the weakening of the wall thickness of the cover 95 at this point, the underside is reinforced with material there. To facilitate the hook-in of the concentrate bag, the surface area between the groove slopes all the way to the front edge. The housing 9 can also decrease its diameter between the lateral surfaces to the front, so that a concentrate bag can be easily pushed on over the housing 9.

1. A cleaning device for a blood treatment device comprising
a base body,
a first and a second line element which extend in the base body from a first surface of the base body,
a first through hole, wherein the first through hole extends from a first point of the first line element spaced apart from the first surface of the base body to a surface of the base body, which differs from the first surface of the base body.

2. The cleaning device according to claim 1,
wherein a connecting duct is formed between the first and the second line element at a second point of the line elements which is spaced apart from the surface of the base body.

3. The cleaning device according to claim 1, wherein
the first through hole extends to a first lateral surface of the base body,
at a third point, which is spaced apart from the first surface of the base body, the second line element has a second through hole to the first lateral surface of the base body, which differs from the first surface of the base body.

4. The cleaning device according to claim 3, wherein
at least one blocking element is attached in the first and second line element, so that no fluidic connection exists between the connecting duct to the first and second through hole, and the blocking element.

5. The cleaning device according to claim 1,
wherein the base body is formed of a first base element and a second base element, wherein the first and second base element in each case have a groove, which has a semicircular cross section, for forming the connecting duct, and the first and second base element in each case have two recesses, which have a semicircular cross section, for forming the first and second line element each when connecting the first and second base element.

6. The cleaning device according to claim 1,
wherein the base body is formed of a first base element and a second base element, and the first base element has two blind bores and the second base element has two through bores, the diameter of which corresponds to the blind bores, for forming the first and second line element,
as well as the first base element and second base element have a groove, which has a semicircular cross section, for forming the connecting duct.

7. The cleaning device according to claim 1, further having
a first cylindrical receptacle comprising at least one through openings and/or a second cylindrical receptacle comprising a through opening, wherein a cylindrical appendage is in each case provided on the through openings, and at least one tubular connecting elements, which can be attached to the appendages and which can be introduced into the connecting duct and/or the through hole in such a way that they connect the receptacles received in the line elements, wherein
the first through hole as well as the connecting duct are open towards the first surface, so that the cylindrical receptacles as well as the tubular connecting elements can be placed into the base body.

8. The cleaning device according to claim 1,
wherein a middle element located between the first and second line element is formed in a curved manner and extends away from the first surface.

9. The cleaning device according to claim 1, further having
at least one sealing shell in the first and/or second line element (2a, 2b), wherein the sealing shell is cylindrical and has at least one lateral passage opening, the diameter of which corresponds to the connecting duct and/or to the through hole and is formed on the jacket surface of the sealing shell (24).

10. A cleaning system for a blood treatment device comprising
a housing for connection to a blood treatment device
the cleaning device of claim 20 which is movably arranged in the housing
a first closing element, by means of which a fluid flow from the first line element can be connected via the first connecting element, and/or a second closing element, by means of which a fluid flow from the second line element can be connected via the second connecting element,
a drive element for moving the cleaning device,
a sensor attached in the housing, for capturing a movement of the cleaning device.

11. The cleaning system according to claim 10, wherein
the sensor is a light barrier on a printed circuit board.

12. The cleaning system according to claim 10, further having
at least one guide rail formed in the housing on which the cleaning device can be shifted, and/or
the first drive element has a spindle nut and an electric motor comprising a threaded spindle.

13. The cleaning system according to claim 10, wherein
the threaded nut is formed on the middle element of the cleaning device, and/or
the middle element has a protrusion, which is formed in the direction of the light barrier, so that a triggering of the light barrier can be attained in response to a moving.

14. Use of a cleaning system according to claim 10 in the case of a blood treatment device.

15. Use of a cleaning device according to claim 1 in the case of a blood treatment device.

16. The cleaning device according to claim 3, wherein at least one blocking element is attached in the first and second line element, so that no fluidic connection exists between the connecting duct to the first and second through hole, and the blocking element has predetermined breaking points.

17. The cleaning device according to claim 1, further having
a first cylindrical receptacle comprising at least two through openings and/or a second cylindrical receptacle comprising a through opening, wherein a cylindrical appendage is in each case provided on the through openings, and
at least two tubular connecting elements, which can be attached to the appendages and which can be introduced into the connecting duct and/or the through hole in such a way that they connect the receptacles received in the line elements, wherein
the first through hole as well as the connecting duct are open towards the first surface, so that the cylindrical receptacles as well as the tubular connecting elements can be placed into the base body.

18. The cleaning device according to claim 1,
wherein a middle element located between the first and second line element is formed in a curved manner and extends away from the first surface perpendicularly.

19. The cleaning device according to claim 1, wherein blood treatment device is a dialysis machine.

20. The cleaning device of claim 1, further comprising a first connecting element, which extends from the base body along the first through hole from the first lateral surface of the base body, so that the first through hole further extends from the first lateral surface of the base body through the first connecting element.
